(19) United States
(12) Patent Application Publication
(10) Pub. No.: US 2023/0211087 A1
(43) Pub. Date: Jul. 6, 2023

(54) NEEDLE-FREE INJECTOR, ASSOCIATED RELOADABLE AND DISPOSABLE NOZZLES, AND METHODS OF INJECTION

(71) Applicant: The Trustees of the University of Pennsylvania, Philadelphia, PA (US)

(72) Inventors: Daniel M. Mazzaferro, Philadelphia, PA (US); Scott P. Bartlett, Philadelphia, PA (US)

(21) Appl. No.: 18/181,226

(22) Filed: Mar. 9, 2023

Related U.S. Application Data
(63) Continuation-in-part of application No. PCT/US2021/049660, filed on Sep. 9, 2021.
(60) Provisional application No. 63/075,857, filed on Sep. 9, 2020.

(51) Int. Cl. A61M 5/30; A61M 5/46; A61M 5/20

(52) U.S. Cl. CPC A61M 5/30; A61M 5/20; A61M 5/46

(57) ABSTRACT

A transdermal injection component, comprising: an injection volume configured to contain an injectable treatment therein, a plunger, the injection volume being in fluid communication with at least one orifice such that the plunger is configured to exert the injectable treatment from the injection volume through the at least one orifice; the plunger sealably engaged with the injection volume, the plunger configured to exert the injectable treatment from the injection volume through the at least one orifice so as to effect transdermal injection of the injectable treatment to a patient; the transdermal injection component being configured to engage with an actuator device configured to effect movement of the plunger to exert the injectable treatment through the at least one orifice.

### CROSS-REFERENCE TO RELATED APPLICATIONS

The present application is a continuation-in-part of International Application No. PCT/US2021/049660, “Needle-Free Injector, Associated Reloadable and Disposable Nozzles, and Methods of Injection” (filed Sep. 9, 2021); which application claims priority to and the benefit of U.S. Pat. Application No. 63/075,857, “Needle-Free Injector, Associated Reloadable and Disposable Nozzles, and Methods of Injection” (filed Sep. 9, 2020). All foregoing applications are incorporated herein by reference in their entireties for any and all purposes.

### TECHNICAL FIELD

The present disclosure relates to the field of needle-free injectors.

### BACKGROUND

Injection of medical treatments has traditionally involved needles, which present issues in terms of handling, safety, and patient discomfort, among other things. Existing needle-free approaches, however, are limited in their application and do not offer fine control over their injection profiles. Accordingly, there is a long-felt need in the art for improved needle-free injection devices, systems, and methods.

### SUMMARY

In meeting the described long-felt needs, the present disclosure first provides a transdermal injection component, comprising: an injection volume configured to contain an injectable treatment therein, a plunger, the injection volume being in fluid communication with at least one orifice such that the plunger is configured to exert the injectable treatment from the injection volume through the at least one orifice; the plunger sealably engaged with the injection volume, the plunger configured to exert the injectable treatment from the injection volume through the at least one orifice so as to effect transdermal injection of the injectable treatment to a patient; the transdermal injection component being configured to engage with an actuator device configured to effect movement of the plunger to exert the injectable treatment through the at least one orifice, and any one or more of: (a) a one-way valve in fluid communication with the sealable injection volume, the one-way valve being configured to place the injection volume into fluid communication with a source of the injectable treatment when the component is engaged with a source of the injectable treatment such that fluid movement is permitted only from the source of the injectable treatment to the injection volume; (b) a sealer disposed so as to seal the sealable injection volume against an environment exterior to the transdermal injection component when (1) the plunger exerts a negative pressure on the injection volume, (2) the component is engaged with a source of the injectable treatment, or both (1) and (2); (c) the component defining an opening disposed proximate to the at least one orifice, the opening being dimensioned such that application of a sufficient negative pressure from the opening encourages patient skin toward the opening; and (d) at least one optionally adjustable element configured to stop a motion of the plunger or a motion of an element engaged with the plunger so as to limit the volume of fluid exerted from the injection volume with motion of the plunger.

In certain aspects, wherein the component comprises a one-way valve in fluid communication with the sealable injection volume, the one-way valve being configured to place the injection volume into fluid communication with a source of the injectable treatment when the component is engaged with the source of the injectable treatment such that fluid movement is permitted only from the source of the injectable treatment to the injection volume.

In certain aspects, the component comprises a sealer disposed so as to seal the sealable injection volume against an environment exterior to the transdermal injection component when (1) the plunger exerts a negative pressure on the injection volume, (2) the component is engaged with a source of the injectable treatment, or both (1) and (2).

In some embodiments, the component comprises an opening disposed proximate to the at least one orifice, the opening being dimensioned such that application of a sufficient negative pressure from the opening encourages patient skin toward the opening. In some embodiments, the opening extends at least partially circumferentially around the at least one orifice.

In certain aspects, the component defines a plurality of openings disposed proximate to at least one of the at least one orifice, the opening being a first opening of the plurality of openings, the plurality of openings being dimensioned such that application of a sufficient negative pressure from the plurality of openings encourages patient skin toward each of the plurality of openings. In some embodiments, the transdermal injection component comprises a surface configured to contact a subject, and wherein (a) the at least one orifice stands proud relative to the surface, (b) wherein the at least one orifice stands flush relative to the surface, or (c) wherein the at least one orifice stands recessed relative to the surface.

In certain aspects, the component comprises at least one element configured to stop a motion of the plunger so as to limit the volume of fluid exerted from the injection volume with motion of the plunger.

In certain aspects, the component comprises a plurality of orifices in fluid communication with the sealable injection volume such that the plunger is configured to exert the injectable treatment from the injection volume through the plurality of orifices.

In certain aspects, the injection volume configured to contain an injectable treatment therein is comprised in an ampoule installed in the transdermal injection component.

The present disclosure also provides a transdermal injection system, comprising: a transdermal injection component as disclosed herein, an actuator device configured to engage with the transdermal injection component, the actuator device comprising a reversibly moveable element configured to effect encouragement of the plunger of the transdermal injection component such that the plunger exerts the injectable treatment through the at least one orifice, the reversibly moveable element being actuated by operation of an electromagnetic field.

In certain aspects, the transdermal injection system further comprises a resilient member configured to exert a force on at least one of the reversibly moveable element and the plunger.

In certain aspects, the transdermal injection system further comprises a source of negative pressure, the source of negative pressure being configured to exert a negative pressure through an opening of the transdermal injection component such that patient skin is exerted generally toward the at least one orifice of the transdermal injection component, optionally such that the patient skin contacts the transdermal injection component.

In certain aspects, the transdermal injection system further comprises an imaging train, the imaging train being configured to determine one or more features of a patient, a location of the injectable treatment, or both.

The present disclosure also provides a method, comprising operating the transdermal injection system disclosed herein so as to effect transdermal injection of the injectable treatment to a patient.

The present disclosure also provides a method comprising: effecting electromagnetically driven motion of a reversibly moveable element so as to drive a plunger engaged with an injection volume that has an amount of an injectable treatment disposed therein, the motion effecting exertion of the injectable treatment through one or more orifices in fluid communication with the injection volume so as to effect transdermal injection of the injectable treatment from the one or more orifices to a patient; and optionally applying a negative pressure to a skin of the patient so as to encourage the skin toward the one or more orifices.

In certain aspects, the method further comprises effecting a series of transdermal injections, the volume of each of the series of transdermal injections optionally being determined by a movable element disposed within the injection volume. In certain aspects, the method further comprises modulating a motion of the plunger in response to a signal collected by an imaging train.

The present disclosure also provides a method, comprising: modulating a flow of a transdermally injected material to a patient in response to a signal collected from an imaging train, the signal being indicative of a location a blood vessel of the patient, the patient’s epidermis, the patient’s dermis, the patient’s fat, the patient’s muscle, the patient’s bone, or any combination thereof.

In certain aspects, the method provides the modulating further being in response to any one or more of a viscosity of the transdermally injected material, a characteristic of the patient’s epidermis, a characteristic of the patient’s dermis, a characteristic of the patient’s fat, a characteristic of the patient’s muscle, and a characteristic of the patient’s bone.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides a schematic view of a system according to the present disclosure;

FIG. 2 provides a cutaway view of a component according to the present disclosure;

FIG. 3 provides a cutaway view of a component according to the present disclosure;

FIG. 4 provides a cutaway view of an injection volume according to the present disclosure in loaded (left panel) and injection (right panel) states;

FIGS. 5A-5G provide cutaway views of a component according to the present disclosure;

FIG. 6 provides a view of a system with an imaging train according to the present disclosure;

FIG. 7 provides a view of a system with an imaging train according to the present disclosure, the imaging train registering the presence of a blood vessel of the patient;

FIGS. 8 and 9 provide a cutaway view of a second end of an injection system, according to an alternative aspect of the present disclosure;

FIG. 10 illustrates a perspective view of a shroud, according to an aspect of the present disclosure;

FIG. 11 provides a cutaway view of a second end of an injection system, according to another alternative aspect of the present disclosure;

FIG. 12 provides a closeup view of the second end of the injection system illustrated in FIG. 11;

FIG. 13 provides a cutaway view of the second end of the injection system shown in FIG. 11 on a user’s skin;

FIG. 14 provides a cutaway view of a second end of an injection system, according to another alternative aspect of the present disclosure;

FIG. 15 shows a graph that illustrates position versus time captured during an injection;

FIG. 16 shows a graph that illustrates force versus time captured during an injection; and

FIG. 17 shows a graph that illustrates current versus time captured during an injection.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” may be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

### Overview

A needle-free intradermal and transdermal device allows the injection of a substance into the dermis of the skin or deeper into the subcutaneous fat, muscle, or periosteal layers. While the term “needle-free” can be used in a device with or without a needle, in both instances no needle is used to first penetrate the skin barrier. There are also multiple types of energy sources to provide the pressure needed to pierce the skin.

The energy sources can include, for example, pressurized gas, cocked spring, electromagnetic (EM) actuator, a combination of these three, or still other energy sources. The devices and methods described herein do not rely on a needle, but rather on pressure to deliver liquids through the skin and into a biological body.

In one aspect, the disclosed technology is a needle-free injection device powered by an electromagnetic actuator and battery that delivers a jet injection at variable depths and volumes with easy reloading for multiple injections using a single or multi-tip disposable syringe. The device can include a single use or rechargeable battery, programmable microcontroller, status light, power switch, multi-button user interface, US probe, and EM actuator to exert force on a plunger. A user can interact with the device by attaching a nozzle filled with liquid to the EM device, turning power on, setting injection depth and volume using a multi-button interface, placing injector on a body surface, allowing US sensing, calculating injection force, and finally pressing the injection button.

One feature of the disclosed technology is the compact, automated, highly customizable, and reloadable EM needle-free injector that delivers cosmetic liquid at variable depths and volumes. The EM actuator can include a stationary magnet and coil assembly. The coil assembly is able to slide relative to the magnet assembly and an adjustable DC/DC boost converter can be driven by a battery to deliver an electrical force in a single axis in either direction. This can exert a force on the plunger, which ultimately transfers the force to a disposable nozzle to inject the liquid. Alternatively, the EM actuator can include a coil assembly that is stationary and a magnet assembly that is able to move relative to the coil assembly.

The disposable single or multi-tip nozzle can be refilled by retracting the plunger using a commutator ring that reverses the power input and polarity of the magnetic coil. As the plunger retracts, liquid can refill the nozzle reservoir through a unique one-way valve connected to an external liquid supply. This disposable nozzle is useful in preventing contamination of the reservoir by allowing to replace nozzles between patients. The plunger can retract to variable lengths and with various power input to inject different depths and volumes. Also, capacitive level sensor or a linear resistive element can be used to confirm fluid flow dynamics and allow appropriate feedback to the computer to drive the EM actuator faster or slower to adjust the injection profile.

The needle-free injection device has utility in injecting any type of liquid, especially cosmetics (neurotoxins, hyaluronic acid fillers, platelet rich plasma, poly-L-lactic acid, calcium hydroxylapatite, biostimulators, fat, fat exosomes, stem cells, cold slurries, hot slurries, and other cosmeceuticals), anesthetics, growth factors, vaccines, oncological treatments, biologics, and other medications.

In another aspect, the disclosed technology includes a reloadable nozzle tip that is disposable and allows for repeat injections, which can also include an open/close one-way valve that moves in conjunction with a plunger at the orifice to allow adequate negative pressure to reload the reservoir. The reloadable nozzle tip can also include a duckbill valve that allows fluid to flow in only one direction (i.e., from the external liquid supply into the nozzle reservoir). The nozzle is a reusable device for each patient and allows reloading for multiple injections in the same patient, but can be disposed of between patients to prevent cross contamination.

One can keep this disposable housing unit separate from the EM actuator device in order to prevent cross contamination. There can be two reservoirs: the first is an internal reservoir within the disposable nozzle and the second is an external reservoir.

The external reservoir can be connected to the internal reservoir through a small channel with a one-way open/close or duckbill valve. This valve allows liquid to flow from the external reservoir to the injectable internal reservoir. This ultimately allows fluid to be easily refilled into the internal reservoir for repetitive injections.

The fluid can either be pushed from the external reservoir or pulled from the internal reservoir when the valve is open. One method of making this possible is by including a cover (i.e., plunger) over the injector nozzle tip while refilling the internal reservoir. This prevents air from entering the injection syringe when attempting to draw fluid from the external reservoir into the internal reservoir. Similarly, this can be achieved by simply using the skin or any other surface to that acts as an artificial cover. By covering the tip, the complete negative pressure is applied on the external reservoir.

The internal reservoir can also be filled from a sealed vial. A variable amount of positive pressure can be optionally introduced into the sealed vial through the use of a separate connector. A variable amount of negative pressure can be exerted on the sealed vial to extract fluid from the vial into the internal reservoir through the use of a separate connector. The fluid can flow through the one-way valve or directly through the disposable nozzle orifice to fill the internal reservoir.

The tubing and open/close one-way valve can be arranged in a way that requires less pressure to evacuate the liquid from the syringe than the air from the distal aspect of the injector nozzle tip. When the valve is closed, the needle-free device can hammer the nozzle plunger down against the internal reservoir only allowing fluid out the needle-free tip for successful injection. The external reservoir can be any commercially made syringe or vial. The injection syringe, one-way valve, and tubing channel can be connected as one piece. A connector piece can be attached to the end of the channel in order to provide a tight fit to various commercially made syringe tips.

A needle-free nozzle can include a vacuum-assisted piping that is connected to a needle-free device to create a seal with the skin to optimize skin contact and seal with the skin, prevent “wet” (failed) injections, increase injection depth, and require a lower force on the hammer/plunger to deliver liquid to a desired depth. The vacuum seal to the skin can be created by a power supply, such as a motor and fan or using the EM actuator. The vacuum seal can be applied directly on the skin surrounding the orifice, on a separate chamber that has been separated from the orifice by a physical barrier included in the shroud, or directly on both chambers including the skin around the orifice and a chamber separated from the orifice. The area of the vacuum chamber(s) can also be changed to alter the amount of pressure being applied on the skin and seal generated on the orifice.

A needle-free injection nozzle can include two or more orifices to inject needle-free fluid, with the potential to use three, four, five, or any number of orifices greater than one to inject the desired fluid can allow for increased dispersion of the fluid with a specific geometric configuration. This can also include one or all of the orifices to be angled at any degree relative to the skin ranging from perfectly perpendicular at 90 degrees, to zero degrees, to 180 degrees. By changing the angle of the orifice relative to the skin surface, the dispersion of the liquid can also be changed. Increase dispersion could be helpful for the filler and vaccines. Vaccines using needle-free devices are shown to mount an increased immune response when compared to traditional needle injections. Additional dispersion using multi tip nozzles can allow further dispersion to potentially mount an even greater response.

These two or more nozzles can be positioned in various orientations or directions to allow for micro amounts of fluid to be injected. As an example, many needle-free single nozzle devices inject approximately 0.1 mL per injection, but these multi-tip needle-free nozzles could still allow for injections of a total of 0.1 mL but with 0.025 mL distributed through four different tips. This allows for greater dispersion and more even distribution. The force of the power supply required to inject through multiple nozzles can accommodate for this change in resistance.

In an aspect, the needle-free injection nozzle can be disposable. The needle-free disposable nozzles that can inject sequential variable volumes allow a preloaded syringe to be injected at variable increments. The nozzle and needle-free device can be connected so that by controlling the needle-free device, a barrier protrudes into the nozzle to only allow the plunger to deliver fluid up to that barrier. Then the barrier can be retracted, the next desired injection volume can be programmed, a new barrier can be set in the nozzle, and the injection delivers the desired volume. For example, after filling a nozzle with 1 mL of fluid, one could first inject 0.1 mL in one location, then 0.2 mL in another location, then 0.4 mL in another location, then 0.3 mL in the final location.

Needle-free disposable nozzles can be configured to allow for engagement between the nozzle plunger and EM hammer (which hammer drives the plunger). The connection can allow for the movement of the EM hammer to be stopped at variable depths, which in turn also stops the movement of the nozzle plunger. The engagement between the EM hammer and the plunger can be effective (i.e., strong enough) to allow for injection speeds in excess of 200 to 300 meters/second without disruption of the engagement. As one example of the foregoing, a barrier can protrude distal to the EM hammer so as to only allow the EM hammer to be delivered by a predetermined distance and then stopped. (The barrier can be moveable or otherwise adjustable, e.g., slidable or retractable). As another example of the foregoing, the EM hammer can be stopped at a desired distance by using a packaged position sensor. When the EM hammer is stopped, so too is the plunger and therefore the liquid from injecting further. In order to ensure the liquid does not continue to leak out due to the initial speed, the EM hammer can also be immediately retracted in the reverse direction after reaching a preset distance, therefore also retracting the plunger and ultimately the liquid. Thus, when the EM hammer stops, the nozzle plunger also stops, thereby preventing additional fluid from being injected. For example, after filling a nozzle with 1 mL of fluid, one could first inject 0.1 mL in one location, then 0.2 mL in another location, then 0.4 mL in another location, then 0.3 mL in the final location.

This disclosure also provides the use of an image-guided (i.e., ultrasound, optical coherence tomography, thermography, or other optical imaging devices) needle-free injection that allows an automated injection to a desired depth by using a set algorithm to determine the required force to inject a particular viscous fluid to a desired depth (i.e., dermis, subcutaneous fat, muscle, bone). A miniaturized ultrasound (US) imaging probe can be included as a packaged sensor module to allow for complete automation of the depth of soft tissue injections and force settings, which can be overridden using the multi-button interface.

Also disclosed is an image-guided (i.e., ultrasound doppler) needle-free injection useful to prevent intravascular injection. An automatic “kill switch” can be included to prevent injections when detecting arteries or veins directly under the needle-free device. This is relevant when injecting hyaluronic acid due to possible complications of tissue necrosis and blindness caused by intravascular injections of filler that embolizes to surrounding tissue.

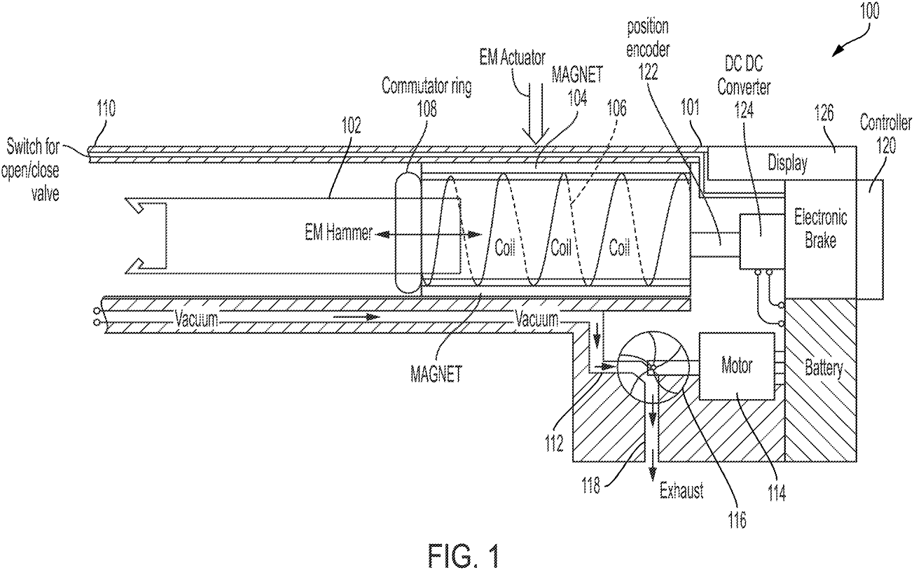

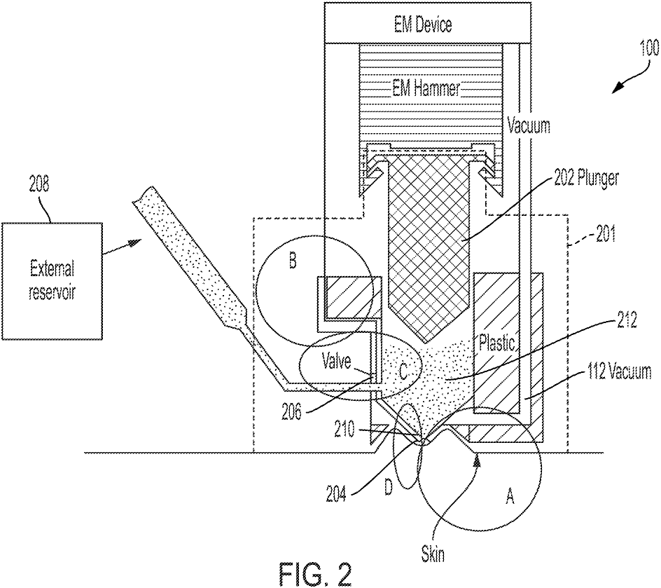

FIGS. 1 and 2 illustrate cutaway views of a first end of an injection system 100 and a second end of the injection system 100, according to aspects of the present disclosure. As shown, the injection system 100 can include an actuator assembly 101 and an injection assembly 201. The actuator assembly can comprise, for example, a voice coil actuator (VCA). The actuator assembly 101 can include an EM hammer element 102, which element 102 is moved via action of a magnet 104 and a coil 106, which operate to move the EM hammer element 102 so as to exert the EM hammer element 102 and move a plunger 202 engaged with the EM hammer element 102. The actuator assembly 101 can also include a commutator ring 108, which ring 108 can be configured to reverse the motion of the EM hammer 102. The actuator assembly 101 can also include a switch 110 or other element that engages with a disposable component and/or a source of injectable treatment, which switch 110 can be configured to permit (or deny) passage of a fluid into and/or out of the disposable component. In an aspect, the actuator assembly 101 excludes the switch 110.

The injection system 100 can also include a vacuum train 112 (e.g., a motor 114, a pump 116, an exhaust/outlet 118, and a controller 120) configured to exert a reduced pressure that encourages a patient’s skin toward a nozzle 204 of the injection assembly 201, and/or stretches the patient’s skin in the vertical or horizontal direction.

The injection system 100 can further include a control train includes, for example, a position encoder 122, a DC-DC converter 124, the controller 120 and/or a controller board (which can be programmable), and other elements. The control train can be configured to modulate a motion of the EM hammer 102 (e.g., the position of the EM hammer 102, the speed of the EM hammer 102) in response to one or more signals (e.g., a signal collected by a sensor or imaging train) or inputs.

The actuator assembly 101 can also include a display 126 and a user interface (not shown). The actuator assembly 101 can also include a battery 128 or otherwise be connected to a power source to provide power to, for example, the motor 114, the controller 120, the position encoder 122, the converter 124, the display 126, or still other components of the actuator assembly 101.

The controller 120 can include software and/or firmware configured to control the injection system 100. A user can program the system 100 through the software and controller 120 to vary a voltage and a current applied to the actuator assembly 101 to adjust the force delivered to the EM hammer 102 and therefore the speed of the EM hammer 102. The actuator assembly 101 can be configured such that an output force applied to the hammer 102 is a function of the current in the coil 106.

In an aspect, the position encoder 122 can comprise a high resolution (e.g., 0.4 micron) linear encoder for position output. The position encoder 122 can provide information to the display 126 to illustrate linear encoder data vs. time to achieve a velocity plot during injection.

A benefit of the actuator assembly 101 described herein is that the user is able to control voltage (i.e., force applied by hammer 102) as inputs and measure displacement and time (i.e., velocity) as outputs. While the velocity plot can be limited by the sampling rate of the linear encoder given the relatively small time interval of the injection (~20-50 ms), the displacement plot is a primary variable of investigation for comparing the software inputs (current, voltage, and the resulting force) to the resulting injection dose success rate, dose variability, and injection depth.

One of the major issues facing needle free injections is limiting “wet shots” where fluid intended to be injected into tissue is not injected, but remains on the surface of the skin. There is a force (pressure) threshold to “open” the skin and allow the fluid to penetrate. This same threshold applies at the end of the injection cycle as the actuator slows to a stop at a given position. In order to limit the time (and displacement) for which the pressure is under this threshold at the end of the injection the actuator must decelerate quickly. This can be accomplished with the actuator assembly 101 by applying a reverse voltage at the end of the intended dose to reduce the force applied (and pressure within an ampoule) from a peak (e.g., 172 N) to zero within approximately 15-20 ms. In another example, the force can be reduced from a threshold of approximately 100 N to zero in 5-10 ms. This limited time (and displacement) between the force threshold needed to penetrate the skin and zero force being applied can significantly reduce the amount of fluid considered a wet shot.

Referring to FIG. 2, the injection assembly 201 can be disposable. The disposable injection assembly 201 is shown within a dashed box, and the injection assembly 201 and injection system 100 being in an injection state.

When the system 100 and the injection assembly 201 are in an injection state, an engagement (shown by circle B) between the component and the system can be configured to actuate a valve 206 to block flow between an external reservoir 208 of injectable treatment and the injection assembly 201. The valve 206 is configured to open and close (e.g., a one-way valve) to allow and prevent fluid communication between the external reservoir 208 of injectable treatment and the injection assembly 201 (shown at circle C). The engagement (shown by circle B) can also cause a movement of a plunger 210 or other closure (or actuation of a valve) that blocks flow between the environment exterior to the nozzle/orifice 204 of an injection volume 212 (shown at circle D).

When in an injection state, the system 100 can be configured so as to block flow between the external reservoir 208 of injectable treatment and allow flow out of the injection volume 212 (driven by the plunger 202). As shown by circle A, the system 100 can also include the vacuum train 112 that applies a reduced pressure to encourage or “tent” a patient’s skin toward the nozzle 204 of the injection assembly 201.

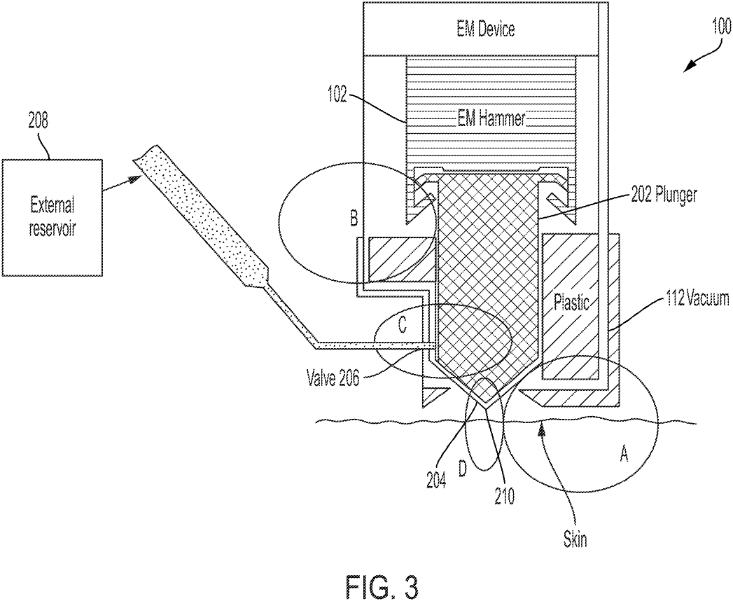

FIG. 3 provides a cutaway view of the injection system 100 shown if FIG. 2, in which view the injection assembly 201 is in a reloading state. As shown, the valve 206 between the external reservoir 208 of injectable treatment is opened (circle C), and the plunger 210 (or valve) blocks flow between the environment exterior to the nozzle/orifice 204 of the injection volume 212 (circle D).

As shown, while the system 100 is in a reloading state, the reduced pressure applied to draw a patient’s skin toward the nozzle 204 of the injection assembly 201 may not be applied (circle A) such that the skin is not “tented” toward the nozzle 204.

Injection assembly 201 (shown at circle C) can also be configured to just allow a one-way flow of liquid (e.g., duckbill valve) from the external reservoir into the injection volume reservoir 212, without letting fluid flow in the opposite direction. This can simplify the valve by eliminating the need for the engagement (circle B).

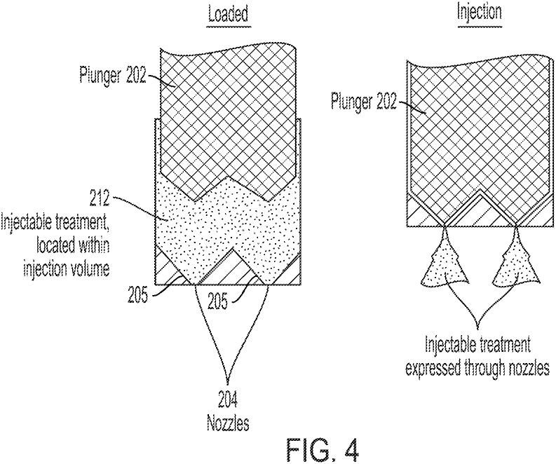

FIG. 4 illustrates a cutaway view of an injection volume 204 according to an aspect of the present disclosure. As shown in the left panel (loaded state), the plunger 202 is positioned within the injection volume 204 such that downward motion of the plunger 202 exerts the injectable treatment within the volume toward the nozzle 204. In an aspect, the nozzle 204 can include a single orifice opening 205 or more than one orifice opening 205. The nozzle 204 can be attached to a housing that contains the injection volume 204, or alternatively, the nozzle 204 can be formed in the housing that contains the injection volume 204.

As shown in the right panel (injection state), downward movement of the plunger 202 exerts injectable treatment through the orifice openings 205 of the nozzle 204.

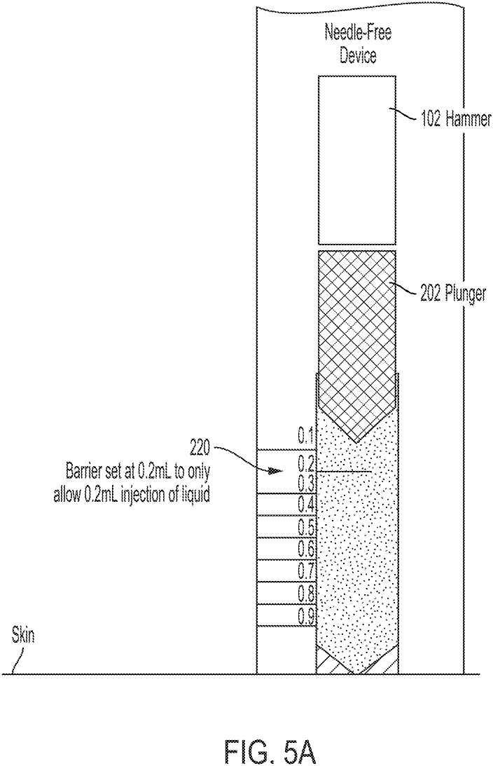

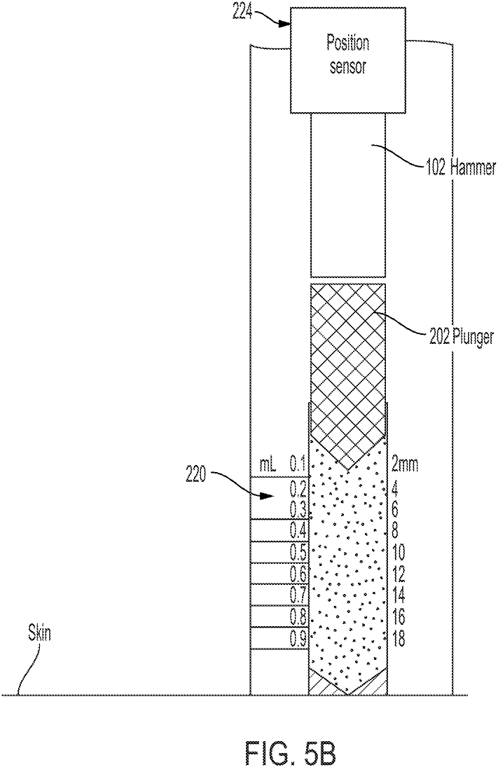

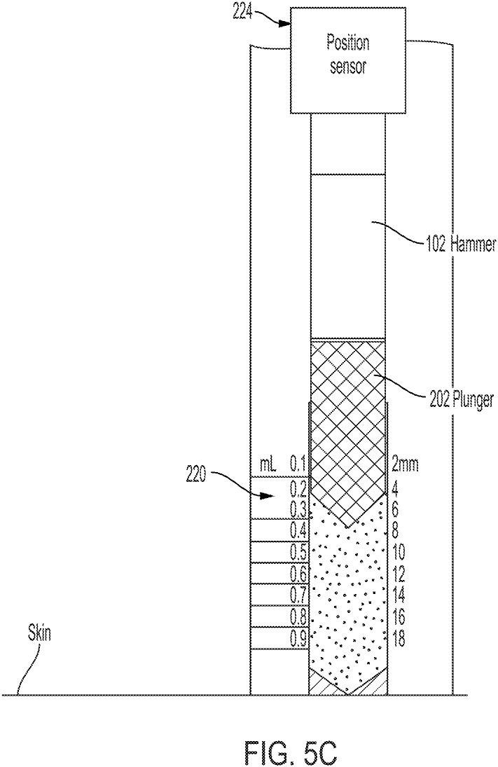

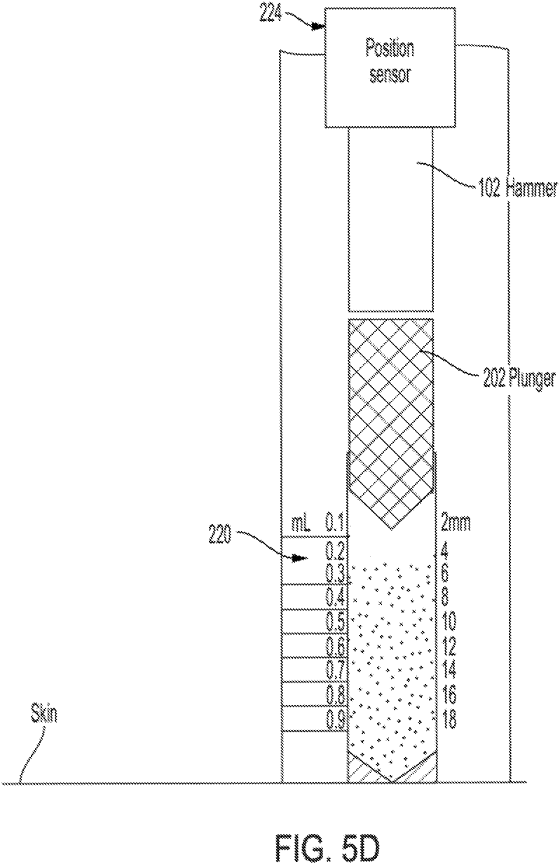

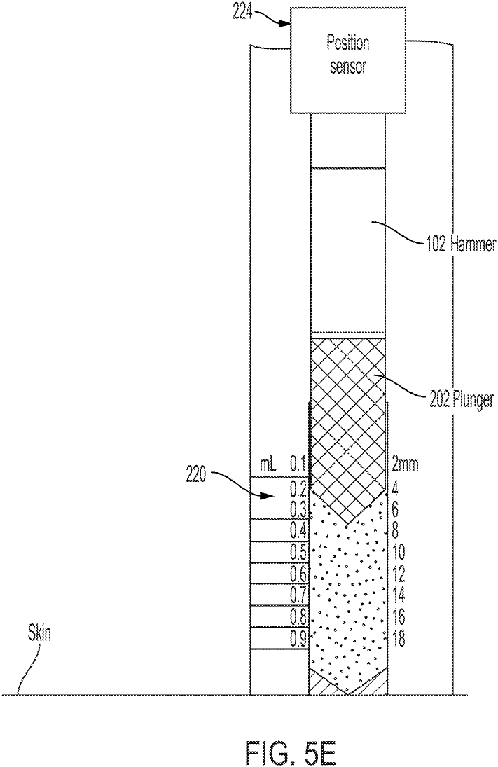

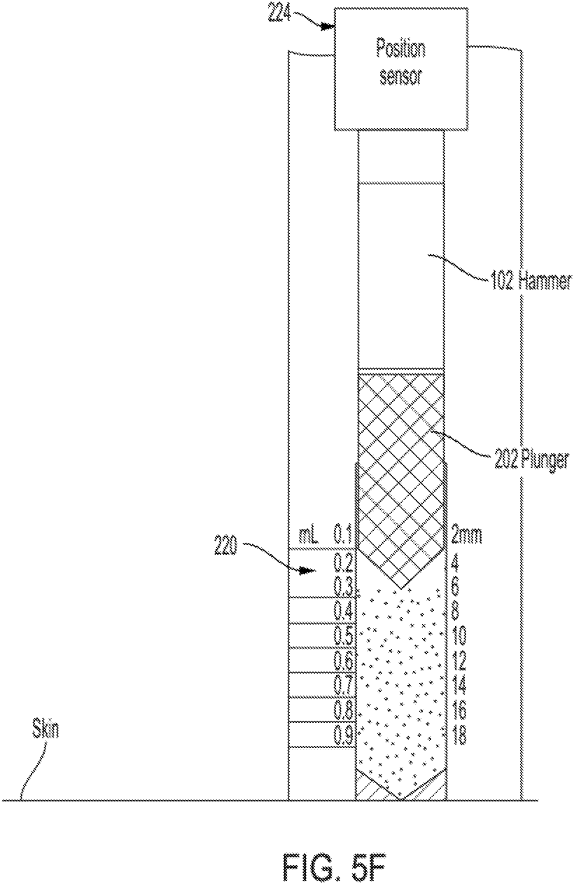

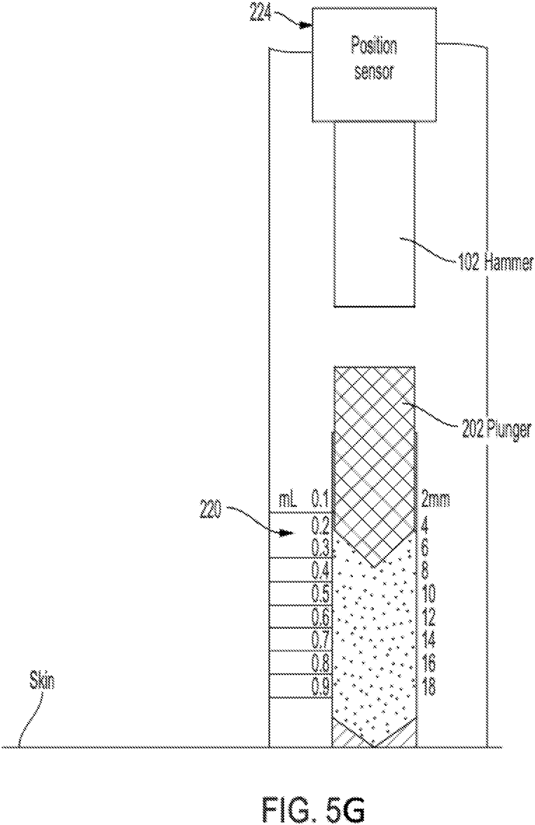

FIG. 5A illustrates a cutaway view of an embodiment that includes a barrier 220 or other element configured to stop the motion of the plunger 202 (and/or the hammer 102 that drives the plunger 202) so as to effect delivery of a pre-set volume of injectable treatment. Such a barrier 220 can be moveable (e.g., from a setting of 0.2 mL to a setting of 0.4 mL). In an aspect, the injection system 100 can include multiple such barriers 220, which barriers 220 can be deployed to set the volume of sequential injections (e.g., 0.2 mL, followed by 0.25 mL, followed by 0.3 mL) delivered by the injection assembly 201. It will be appreciated that the sequential injections can be delivered without the barrier 220. For example, the force applied by the plunger 202 and the time for which that force is applied can be controlled during the course of the injection.

FIGS. 5B through 5G illustrate cutaway views of a sequence of injections using reversibility of the plunger 202. The injection system 100 can also include a position sensor 224 configured to detect a position of the hammer 102 and/or the plunger 202. Referring to FIGS. 5B and 5C, the position sensor 224 can allow the hammer 102 and the plunger 202 to administer liquid (e.g., 0.2 mL) by allowing each of them to move forward a certain distance (e.g., 4 mm) that equates to a desired volume. As shown, movement of the hammer 102 and/or the plunger 202 by a distance of 4 mm delivers 0.2 mL of liquid. The movement of the hammer 102 can be coupled with the movement of the plunger 202. For example, retracting the hammer 102 can result in a coordinated and/or coupled retraction of plunger 202. Alternatively, the movement of the hammer 102 can be independent of the movement of the plunger 202. For example, the hammer 102 can retract without drawing up the plunger 202. Referring to FIG. 5D, after the liquid is administered, the hammer 102 and the plunger 202 are retracted back to a starting position. Referring to FIG. 5E, in an additional or alternative aspect, the hammer 102 and the plunger 202 remain stopped after the liquid is administered. Referring to FIG. 5F, in an additional or alternative aspect, the hammer 102 and the plunger 202 are retracted by a set amount (e.g., 2 mm) after the liquid is administered. Referring to FIG. 5G, in a further additional or alternative aspect, the hammer 102 is retracted by a set amount (e.g., 2 mm) while the plunger 202 remains stationary or, alternatively, is retracted by a lesser amount. It should be understood, however, that hammer 102 and plunger 202 need not be coupled to one another. As shown in, e.g., FIGS. 5A - 5G, hammer 102 and plunger 202 can be separated by a space, which space affords the hammer an opportunity to reach to a non-zero velocity (and even accelerate, in some embodiments) as the hammer is encouraged toward plunger 202. Without being bound to any particular theory or embodiment, affording space to allow the hammer to achieve a non-zero velocity before the hammer contacts the plunger can result in improved device performance. It should be understood, however, that a space between hammer 102 and plunger is not a requirement.

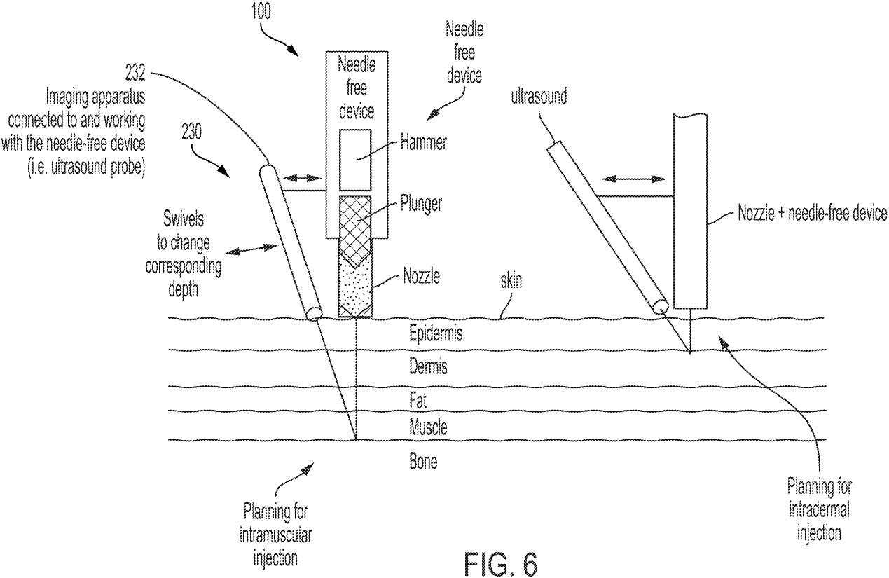

FIG. 6 illustrates a schematic of the injection system 100 that includes an imaging train 230, according to an aspect of this disclosure. As shown in the left panel, the imaging train 230 can include a probe 232 (e.g., an ultrasound probe, locating the depth of the blood vessel of the patient) that is moveable or otherwise adjustable to change the depth of the probe’s 232 monitoring.

The probe 232 can be configured to, e.g., detect a depth of a patient’s skin/epidermis, dermis, fat, muscle, or even bone. The imaging train 230 can then provide a signal or other information to the actuator assembly 101, from which signal or other information regarding the depth of injection of treatment from the injection system 100 is determined.

For example, based on the desired depth of injection (e.g., to the dermis depth), the injection system 100 can determine the necessary velocity and/or force needed to be exerted on the EM hammer and thus the plunger in order to deliver the injectable treatment to that depth, and accordingly then modulate the motion of the hammer 102 that drives the plunger 202, so as to achieve the desired fluid dynamics.

A user can assemble a library of injection depth settings (e.g., based on experiments or other data, a library of fluid force needed to penetrate to various depths, a library of the fluid force needed to penetrate to the depth of muscle or other tissue in a typical patient having a given age, a given weight, or other characteristics, and the like), and then use the signal gathered by the imaging train to select, from the library, the appropriate setting needed for a given injection. The settings can also be based on the location (e.g., face, arm, leg) to which the user wishes to deliver the injectable treatment. In this way, the disclosed technology can be adapted by the user for use on different parts of a subject, whereas many existing needle-less injection systems are configured with pre-set settings and thus are useful only for the part of the body that the pre-set settings correspond to. For this reason, an existing needle-less injector that is pre-set to deliver a treatment to the muscle of a patient’s arm may not be suitable for delivering a cosmetic agent to a patient’s face, as the settings configured to deliver a treatment to arm muscle may not be suitable for delivering a cosmetic treatment only to a patient’s facial skin, as using the arm muscle settings on the face would result in the treatment being delivered far below the face skin potentially putting the critical facial structure at risk of injury (e.g. blood vessels and nerves).

As but one example, a user who desires to inject a treatment to a patient’s dermis can use the imaging train 230 to determine the depth of the patient’s dermis and then, by reference to a library of fluid velocities/forces, determine the fluid velocity needed to achieve injection to the depth of the patient’s dermis. The right panel of FIG. 6 provides a view of the system 100 with the imaging train 230 arranged to collect information at the depth of a patient’s dermis.

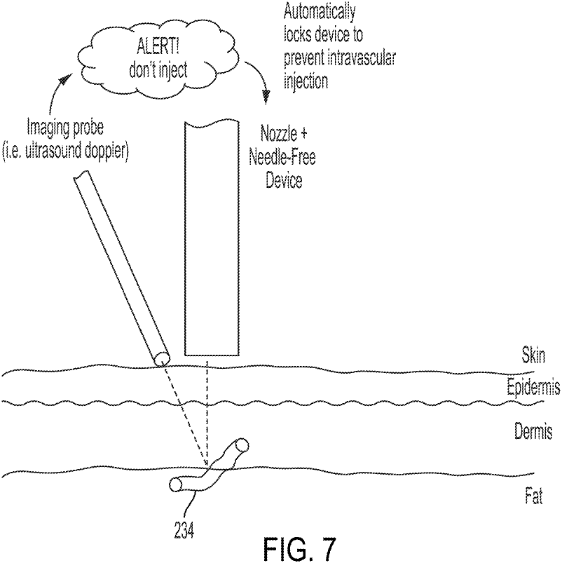

FIG. 7 provides a schematic of the imaging train 230 being used to locate a blood vessel within a patient. Without being bound to any particular theory, a user may wish to avoid injecting injectable treatment material into a patient’s blood vessel 234.

As shown in FIG. 7, the imaging train 230 can be used to locate a blood vessel within a patient and – if the blood vessel 234 (shown by the tubular, worm-like element that is detected by the train 230) lies within the expected pathway of injection – alert the user and even be configured to lock (e.g., by immobilizing the hammer 102 that presses on the plunger 202, by engaging a valve (not shown) that blocks fluid flow out of the injection volume 212) or otherwise operate the system 100 to prevent injection into the blood vessel 234.

Without being bound to any particular theory or embodiment, such a feature can increase patient safety and ease of use for the operator.

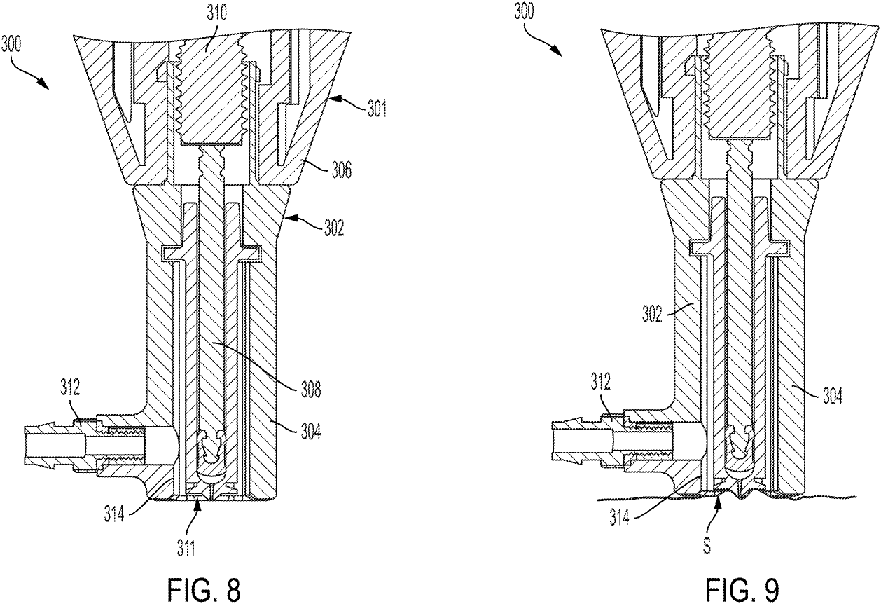

FIGS. 8 and 9 illustrate cutaway views of a second end of an injection system 300, according to an alternative aspect of the present disclosure. It will be appreciated that the injection system 300 can be operated and configured in a substantially similar manner as the injection system 100 described herein unless otherwise described below. The injection system 300 comprises an actuator assembly 301 and an injection assembly 302. The injection assembly 302 comprises a shroud 304 that is removably coupled to a nose 306 of the actuator assembly 301. The coupling can be, e.g., a LUER-LOCK™ or other coupling, such as a screw or bayonet coupling. The injection system 300 further comprises an ampoule 308 that can engage with hammer 310, although this is not a requirement, as the ampoule can also engage with another component of the injector system and the ampoule need not couple to hammer 310. The ampoule 308 can include a plunger, as shown. As one example, the ampoule can engage with the injector system such that hammer 310 and the plunger of the ampoule are independent of one another; i.e., that the hammer and plunger are free of being coupled to one another. The ampoule 308 can be slid into and then secured into the shroud 304, e.g., by insertion and rotation. A height of the ampoule 308 relative to skin of a patient can be adjusted based on a desired injection depth. The ampoule 308 can be sterilizable and refillable. That is, the ampoule 308 can be suited for more than one use or repeated uses. Alternatively, the ampoule 308 can be disposable. That is, ampoule 308 can be suited for single-use. The shroud 304 can also be disposable. It should be understood that ampoule 308 can be prefilled with an injectable treatment and then installed into shroud 304. In some embodiments, ampoule 308 can be installed into shroud 304 and then filled.

The injection assembly 302 further comprises a vacuum nozzle 312 coupled to the shroud 304; the injection nozzle can connect to a side-arm (not labeled) of shroud 304. The vacuum nozzle 312 defines a nozzle channel therethrough that fluidly connects a vacuum source (e.g., via the vacuum chain 112) to a vacuum channel 314 defined by the shroud 304. The vacuum channel 314 can extend circumferentially about an injection opening 311 of the shroud 304. In an aspect, the vacuum channel 314 opens to the injection opening 311. A vacuum tube can be connected to the nozzle 312 to form a substantially airtight seal between the vacuum source on a patient’s skin S. As illustrated in FIG. 9, the patient’s skin S is tented up due to the vacuum source applied through the vacuum channel 312. In this aspect, the skin S directly around the needle-free ampoule 308 orifice is tented up, which can create an optimal seal. The tented-up skin results in improved depth of injection of the fluid and decreased wet or failed injections when vacuum suction is applied. For example, the injection depth can increase by approximately 2.0 - 2.5x when compared to an injection when no vacuum suction is applied. Another benefit of the vacuum suction is that a lower load force applied by a user is required (e.g., axial load force or push down force) to achieve the desired injection depth. Further, the vacuum suction ensures a tighter seal to optimize the injection of the liquid entering the skin, thereby decreasing, and potentially eliminating, the risk of wet or failed injections.

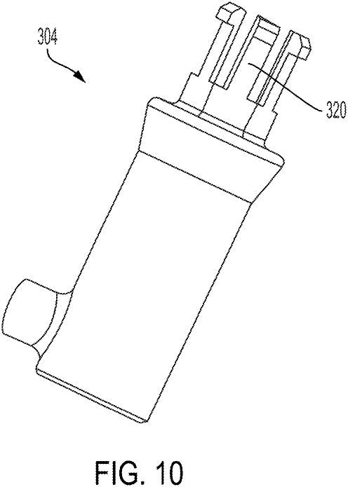

FIG. 10 illustrates a perspective view of the shroud 304, according to an aspect of this disclosure. The shroud 304 can include finger elements 320 that facilitate the connection and removal of the shroud 304 to the nose 306 of the actuator assembly 301. The finger elements 320 can be flexible, such that during connection, the finger elements 320 flex radially inward until the shroud 304 is fully inserted into the nose 306. After the shroud 304 has been fully inserted, the finger elements 320 flex radially outward to removably lock (e.g., snap-fit) the shroud 304 to the nose 306.

The injection assembly can include a sterile, disposable shroud 304 and a sterile, disposable ampoule 308. The sterile, disposable ampoule 308 can be pre-filled, but can also be sterilizable and re-fillable. The ampoule 308 can include a single needle-free opening. Alternatively, the ampoule 308 can include more than one needle-free opening. For example, the ampoule 308 can include at least 2, 3, 4, 5, 6, 7, 8, 9, 10, or more than 10 openings. As described elsewhere herein, the injection assembly including the sterile, disposable shroud 304 and the sterile, disposable ampoule 308 can be provided as a kit wherein the sterile, disposable, pre-filled, or optionally refillable ampoule 308 can be loaded into the sterile, disposable shroud 304. The assembled disposable injection system can then be connected to the actuator assembly and a vacuum source. In some embodiments, the shroud and ampoule are integrated together, i.e., the ampoule is comprised within the shroud and is not otherwise insertable or removeable. In this way, an integrated assembly can include a pre-filled ampoule; an integrated assembly can also include an unfilled ampoule that is then filled before use. Following completion of one or more injections, the injection assembly 302 can be disconnected from the actuator assembly 301 and the vacuum source. The disconnected injection assembly 302 can then be discarded, and a new injection assembly can be installed. Alternatively, the disconnected injection assembly 302 can be further disassembled so that the spent ampoule 308 is removed from the shroud 304 and the shroud 304 is disposed of, and the ampoule is optionally sterilized and refilled for reloading into a new, sterile, disposable shroud 304. Filling of the ampoule, refilling of the ampoule, or both can be accomplished by a one-way valve approach in which a one-way valve permits flow of the injectable treatment into the ampoule, e.g., in response to withdrawing the plunger of the ampoule. A one-way valve can be incorporated into the ampoule; a one-way valve can also be incorporated into the shroud.

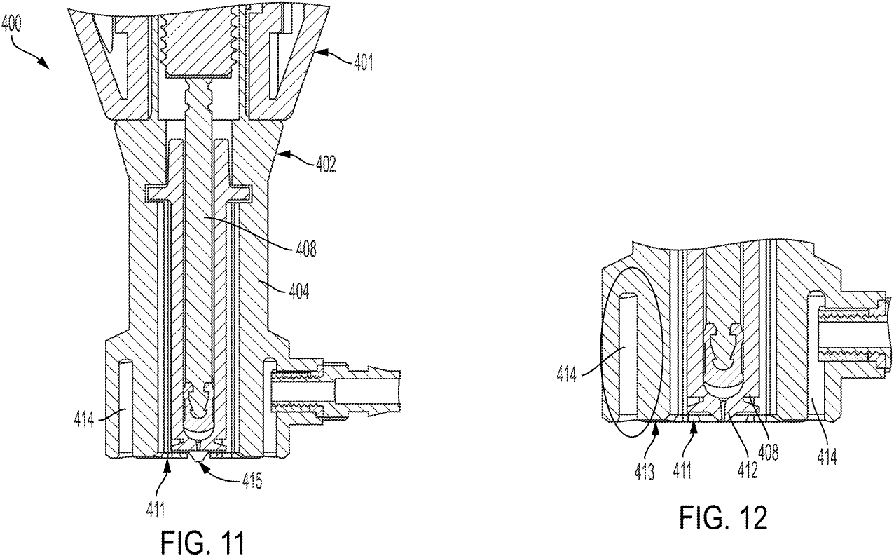

FIGS. 11-13 illustrate cutaway views of a second end of an injection system 400, according to another alternative aspect of the present disclosure. It will be appreciated that the injection system 400 can be operated and configured in a substantially similar manner as the injection systems 100 and 300 described herein unless otherwise described below. The injection system 400 comprises an actuator assembly 401 and an injection assembly 402. The injection assembly 402 comprises a shroud 404 that is removably coupled to the actuator assembly 401. The shroud 404 defines a vacuum channel 414 defined therein. With reference to FIG. 12, which illustrates a close-up view of a bottom end of the injection assembly 402, the vacuum channel 414 can extend circumferentially about an injection opening 411 of the shroud 404. The vacuum channel 414 is spaced radially outward from the injection opening 411. In this aspect, the vacuum channel 414 is separated from (e.g., isolated) from a tip of an ampoule 408. For example, a plastic type component 413 can be positioned between the vacuum channel 414 and the opening 411. The plastic type component 413 (or component made of any other type of material) can range from extremely thin to extremely thick to change the distance between the injection opening and the vacuum channel.

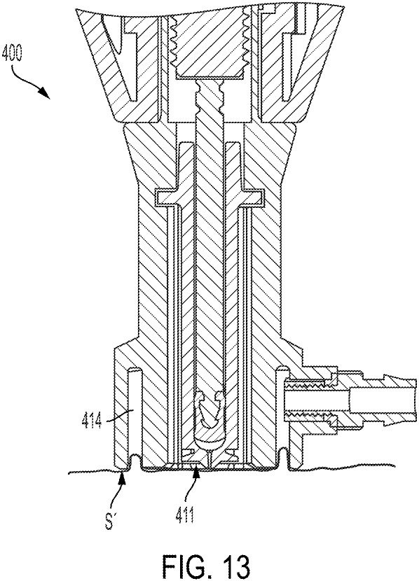

With reference to FIG. 13, a vacuum can be applied through the vacuum channel 414 to tent-up a user’s skin S′ immediately surrounding the tip of the ampoule 408. In this aspect, the user’s skin S′ does not tent-up directly under the tip of the ampoule 408 itself. Instead, the user’s skin S′ tents-up at a distance spaced radially outward from the tip of the ampoule 408. In doing so, the skin S′ that immediately surrounds the tented skin under the vacuum channel 414 is stretched in the horizontal direction to change the injection depth, reliably preventing wet or failed injection, and affect dispersion of liquid.

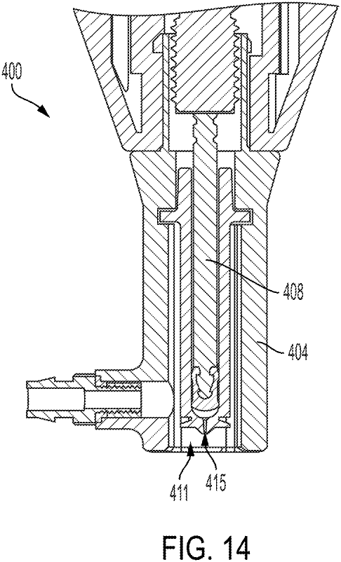

With reference to FIG. 11, a tip 415 of the ampoule 408 of the injection system 400 can extend through the opening 411 of the shroud 404 (e.g., slightly projecting -proud). During use, the tip 415 projects proud relative to an opening of the vacuum channel 414 in the shroud 404, such that the ampoule 408 comes in contact with the skin S′ prior to the shroud 404. In an aspect, the tip 415 can extend any distance from the bottom end of the shroud 404. In an aspect, the tip 425 can extend at least 2.0 mm beyond the bottom end of the shroud 404. The shroud 404 can comprise an elastomeric material, such that during injection when the negative pressure is applied by the vacuum to the skin, the shroud 404 retracts slightly projecting the tip 415 proud relative to the opening of the vacuum channel 414. Alternatively, and with reference to FIG. 14, tip 415 can be inward relative to the opening 411 of the shroud 404 (e.g., recessed). In this aspect, the shroud 404 can contact the skin S′ prior to (or instead of) the tip 415 of the ampoule 408. It will be appreciated that the shroud 404 can include multiple openings (i.e., plurality of openings) to one or more vacuum channels 414. In an aspect, at least one of the plurality of openings is proximate to each one of the at least one orifices defined by a nozzle 412. In an aspect, a plurality of openings can be proximate to the at least one orifice. In another aspect, each of the at least one orifices can include a single one of the plurality of openings proximate to the at least one orifice.

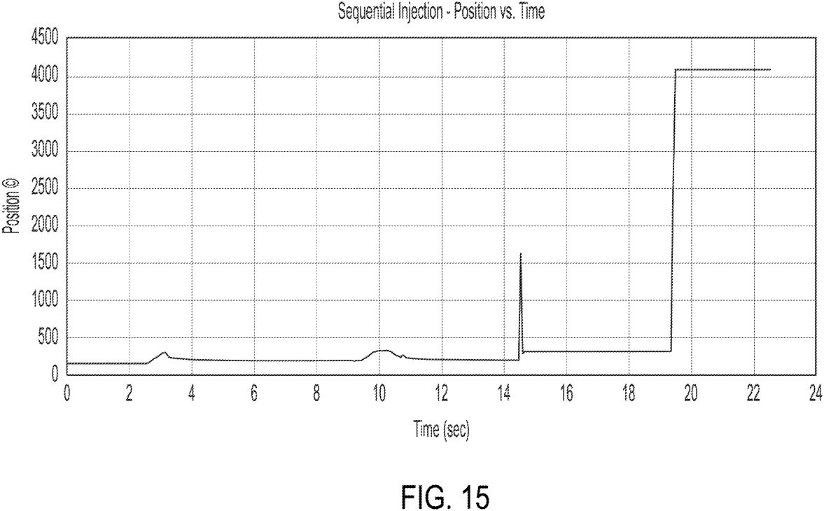

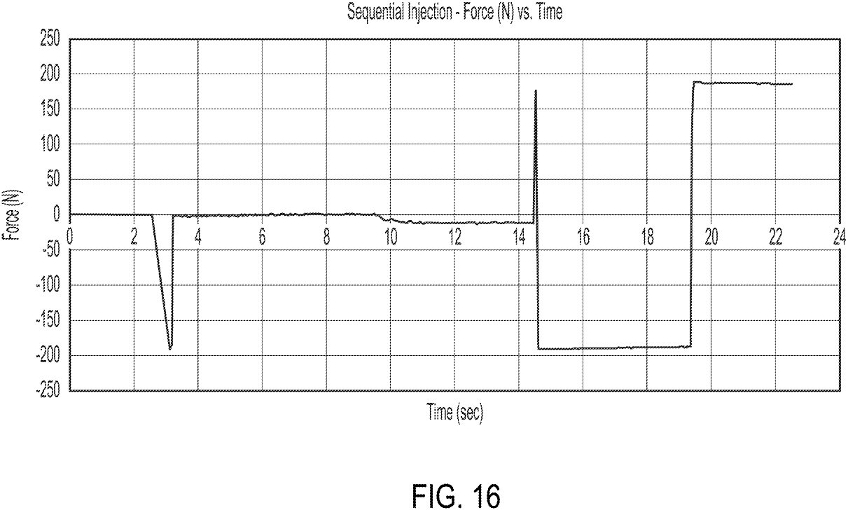

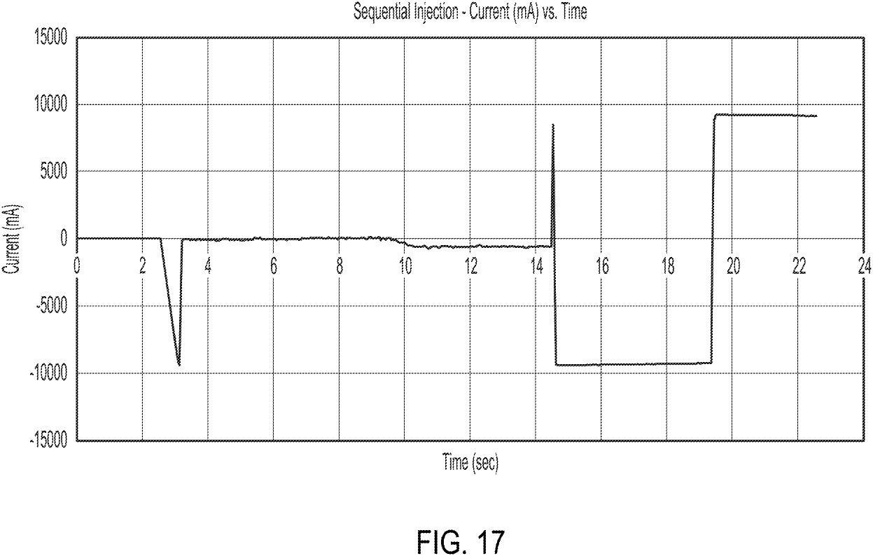

FIGS. 15-17 show graphs that illustrate a profile of position, force, and current over time, respectively, captured during a sequence of two injections, according to an aspect of this disclosure. FIGS. 15-17 are intended to be illustrative only of a particular embodiment of an injection sequence and not limiting. Other alternative injection sequences are therefore contemplated. The first of the two injections includes 0.15 mL, and the second of the two injections includes 0.3 mL. The sequence of injections can be described as follows:
a. Seconds 2-4: The actuator piston is retracted to allow for insertion of the loaded ampoule.
b. Seconds 10-14: The injector is moved from a horizontal position to a vertical one to prepare for the injection. The force necessary to maintain a constant position increases slightly as it’s now supporting the weight of the VCA piston. Force becomes slightly higher (negative) to hold the piston in place.
c. Seconds 14-19: Actuator advances to perform the first injection then immediately returns to its starting position.
d. Seconds 19-22: Actuator advances to perform the second injection and maintains its position after completing the injection.

### Aspects

The following Aspects are illustrative only and do not limit the scope of the present disclosure or the appended claims. Any part or parts of any one or more Aspects can be combined with any part or parts of any one or more other Aspects.

Aspect 1. A transdermal injection component, comprising:
an injection volume configured to contain an injectable treatment therein,
a plunger,
the injection volume being in fluid communication with at least one orifice such that the plunger is configured to exert the injectable treatment from the injection volume through the at least one orifice;
the plunger sealably engaged with the injection volume,
the plunger configured to exert the injectable treatment from the injection volume through the at least one orifice so as to effect transdermal injection of the injectable treatment to a patient;
the transdermal injection component being configured to engage with an actuator device configured to effect movement of the plunger to exert the injectable treatment through the at least one orifice, and any one or more of:
(a) a one-way valve in fluid communication with the sealable injection volume,
the one-way valve being configured to place the injection volume into fluid communication with a source of the injectable treatment when the component is engaged with a source of the injectable treatment such that fluid movement is permitted only from the source of the injectable treatment to the injection volume;
(b) a sealer disposed so as to seal the sealable injection volume against an environment exterior to the transdermal injection component when (1) the plunger exerts a negative pressure on the injection volume, (2) the component is engaged with a source of the injectable treatment, or both (1) and (2);
(c) the component defining an opening disposed proximate to the at least one orifice, the opening being dimensioned such that application of a sufficient negative pressure from the opening encourages patient skin toward the opening; and
(d) at least one optionally adjustable element configured to stop a motion of the plunger or a motion of an element engaged with the plunger so as to limit the volume of fluid exerted from the injection volume with motion of the plunger.

Aspect 2. The transdermal injection component of Aspect 1, wherein the component comprises a one-way valve in fluid communication with the sealable injection volume, the one-way valve being configured to place the injection volume into fluid communication with a source of the injectable treatment when the component is engaged with the source of the injectable treatment such that fluid movement is permitted only from the source of the injectable treatment to the injection volume.

Aspect 3. The transdermal injection component of Aspect 1, wherein the component comprises a sealer disposed so as to seal the sealable injection volume against an environment exterior to the transdermal injection component when (1) the plunger exerts a negative pressure on the injection volume, (2) the component is engaged with a source of the injectable treatment, or both (1) and (2).

Aspect 4. The transdermal injection component of Aspect 1, wherein the component comprises an opening disposed proximate to the at least one orifice, the opening being dimensioned such that application of a sufficient negative pressure from the opening encourages patient skin toward the opening.

Aspect 5. The transdermal injection component of Aspect 4, wherein the opening extends at least partially circumferentially around the at least one orifice.

Aspect 6. The transdermal injection component of Aspect 1, wherein the component defines a plurality of openings disposed proximate to at least one of the at least one orifice, the opening being a first opening of the plurality of openings, the plurality of openings being dimensioned such that application of a sufficient negative pressure from the plurality of openings encourages patient skin toward each of the plurality of openings.

Aspect 7. The transdermal injection component of Aspect 1, wherein the transdermal injection component comprises a surface configured to contact a subject, and wherein (a) the at least one orifice stands proud relative to the surface, (b) wherein the at least one orifice stands flush relative to the surface, or (c) wherein the at least one orifice stands recessed relative to the surface.

Aspect 8. The transdermal injection component of Aspect 1, wherein the component comprises at least one element configured to stop a motion of the plunger so as to limit the volume of fluid exerted from the injection volume with motion of the plunger.

Aspect 9. The transdermal injection component of Aspect 1, wherein the component comprises a plurality of orifices in fluid communication with the sealable injection volume such that the plunger is configured to exert the injectable treatment from the injection volume through the plurality of orifices.

Aspect 10. The transdermal injection component of Aspect 1, wherein the injection volume configured to contain an injectable treatment therein is comprised in an ampoule installed in the transdermal injection component.

Aspect 11. A transdermal injection system, comprising:
a transdermal injection component according to Aspect 1;
an actuator device configured to engage with the transdermal injection component,
the actuator device comprising a reversibly moveable element configured to effect encouragement of the plunger of the transdermal injection component such that the plunger exerts the injectable treatment through the at least one orifice,
the reversibly moveable element being actuated by operation of an electromagnetic field.

Aspect 12. The transdermal injection system of Aspect 11, further comprising a resilient member configured to exert a force on at least one of the reversibly moveable element and the plunger.

Aspect 13. The transdermal injection system of Aspect 11, further comprising a source of negative pressure,

The source of negative pressure being configured to exert a negative pressure through an opening of the transdermal injection component such that patient skin is exerted generally toward the at least one orifice of the transdermal injection component, optionally such that the patient skin contacts the transdermal injection component.

Aspect 14. The transdermal injection system of Aspect 10, further comprising an imaging train, the imaging train being configured to determine one or more features of a patient, a location of the injectable treatment, or both.

Aspect 15. A method, comprising operating the transdermal injection system of Aspect 11 so as to effect transdermal injection of the injectable treatment to a patient.

Aspect 16. A method, comprising:
effecting electromagnetically driven motion of a reversibly moveable element so as to drive a plunger engaged with an injection volume that has an amount of an injectable treatment disposed therein,
the motion effecting exertion of the injectable treatment through one or more orifices in fluid communication with the injection volume so as to effect transdermal injection of the injectable treatment from the one or more orifices to a patient; and
optionally applying a negative pressure to a skin of the patient so as to encourage the skin toward the one or more orifices.

Aspect 17. The method of Aspect 16, further comprising effecting a series of transdermal injections, the volume of each of the series of transdermal injections optionally being determined by a movable element disposed within the injection volume.

Aspect 18. The method of Aspect 16, further comprising modulating a motion of the plunger in response to a signal collected by an imaging train.

Aspect 19. A method, comprising:
modulating a flow of a transdermally injected material to a patient in response to a signal collected from an imaging train,
the signal being indicative of a location a blood vessel of the patient, the patient’s epidermis, the patient’s dermis, the patient’s fat, the patient’s muscle, the patient’s bone, or any combination thereof.

Aspect 20. The method of Aspect 19, the modulating further being in response to any one or more of a viscosity of the transdermally injected material, a characteristic of the patient’s epidermis, a characteristic of the patient’s dermis, a characteristic of the patient’s fat, a characteristic of the patient’s muscle, and a characteristic of the patient’s bone.

### Additional Disclosure

Also provided is a transdermal injection component, comprising: a sealable injection volume configured to contain an injectable treatment therein; a plunger, the plunger sealably engaged with the injection volume, the plunger optionally being configured to engage with an element of an actuator device, and the plunger configured to exert the injectable treatment from the injection volume so as to effect transdermal injection of the injectable treatment to a patient; and
(a) a one-way valve in fluid communication with the sealable injection volume,
the one-way valve being configured to place the injection volume into fluid communication with a source of the injectable treatment when the component is engaged with a source of the injectable treatment such that fluid movement is permitted only from the source of the injectable treatment to the injection volume;
(b) a sealer disposed so as to seal the sealable injection volume against the environment exterior to the transdermal injection component when (1) the plunger exerts a negative pressure on the injection volume, (2) the component is engaged with a source of the injectable treatment, or both (1) and (2);
(c) the component defining an opening disposed proximate to the at least one orifice, the opening being dimensioned such that application of a sufficient negative pressure from the opening encourages patient skin toward the opening;
(d) at least one optionally adjustable element configured to stop a motion of the plunger or a motion of an element engaged with the plunger so as to limit the volume of fluid exerted from the injection volume with motion of the plunger;
(e) the component comprising a plurality of orifices in fluid communication with the sealable injection volume such that the plunger is configured to exert the injectable treatment from the injection volume through the plurality of orifices; or
two or more of (a), (b), (c), (d), and (e) in any combination.

A component can include a one-way valve in fluid communication with the sealable injection volume, the one-way valve being configured to place the injection volume into fluid communication with a source of the injectable treatment when the component is engaged with the source of the injectable treatment such that fluid movement is permitted only from the source of the injectable treatment to the injection volume.

A component can also include a sealer disposed so as to seal the sealable injection volume against the environment exterior to the transdermal injection component when (1) the plunger exerts a negative pressure on the injection volume, (2) the component is engaged with a source of the injectable treatment, or both (1) and (2).

A component can be arranged so as to define an opening disposed proximate to the at least one orifice, the opening being dimensioned such that application of a sufficient negative pressure from the opening encourages patient skin toward the opening.

A component can include at least one element configured to stop a motion of the plunger so as to limit the volume of fluid exerted from the injection volume with motion of the plunger.

A component can include a plurality of orifices in fluid communication with the sealable injection volume such that the plunger is configured to exert the injectable treatment from the injection volume through the plurality of orifices.

Further provided is a transdermal injection system, comprising:
a transdermal injection component according to the present disclosure;
an actuator device configured to engage with the transdermal injection component,
the actuator device comprising a reversibly moveable element configured to engage with the plunger of the transdermal injection component so as to effect motion of the plunger,
the reversibly moveable element being actuated by operation of an electromagnetic field.

The transdermal injection system can include a resilient member configured to exert a force on at least one of the reversibly moveable element and the plunger.

The transdermal injection system can also include a source of negative pressure, the source of negative pressure being configured to exert a negative pressure such that patient skin is exerted generally in the direction of the transdermal injection component, optionally such that the patient skin contacts the component.

The transdermal injection system can include an imaging train, the imaging train being configured to determine one or more features of a patient, a location of the injectable treatment, or both. It should be understood that the imaging train (or a portion thereof) can be incorporated into a component, but can also be incorporated into the actuator device or other element with which the component engages. The imaging train can be configured to locate a blood vessel within the patient.

The imaging train can be configured to locate one or more of a patient’s epidermis, dermis, fat, muscle (including various layers of muscle), or bone.

The imaging train comprises at least one of an ultrasound probe, an optical coherence tomography probe, or a thermography probe, or one or more other types of optical imaging devices.

The transdermal injection system can be configured to modulate a flow of the injectable treatment in response to a signal collected from the imaging train.

The transdermal injection system can be configured to restrict a flow of the injectable treatment in response to a signal collected from the imaging train that is indicative of a blood vessel within the patient.

Also provided is a method, comprising operating the transdermal injection system of the present disclosure so to as to effect transdermal injection of the treatment to a patient.

Further provided is a method, comprising: effecting electromagnetically driven motion of a reversibly moveable element so as to drive a plunger engaged with an injection volume that has an amount of an injectable treatment disposed therein, the motion effecting exertion of the injectable treatment through a plurality of orifices in fluid communication with the injection volume so as to effect transdermal injection of the injectable treatment from the plurality of orifices to a patient; and optionally applying a negative pressure to the skin of the patient so as to encourage the skin toward the plurality of orifices.

The method can further include effecting a series of transdermal injections, the volume of each of the series of transdermal injections being determined by a movable element disposed within the injection volume. Each of the series of injections can be of the same volume at same or different depths, but this is not a requirement, as one or more of the series of injections can be of a different volume at the same or different depths than the others.

The method can further comprise modulating a motion of the plunger in response to a signal collected by an imaging train.

Also provided is a method, comprising: modulating a flow of a transdermally injected material to a patient in response to a signal collected from an imaging train, the signal being indicative of a location a blood vessel of the patient, the patient’s epidermis, the patient’s dermis, the patient’s fat, the patient’s muscle, the patient’s bone, or any combination thereof. The modulating can be in response to a viscosity of the transdermally injected material. The viscosity of the transdermally injected material can be characterized as G′ (or other rheologic properties), in some instances. The signal can be, e.g., an ultrasonic signal or an infrared signal. The imaging train can collect signals at two or more depths within the patient.

The modulating is at least partially in response to one or more estimated characteristics of the patient’s epidermis, the patient’s dermis, the patient’s fat, the patient’s muscle, the patient’s bone, or any combination thereof. The modulating can also be effected by modulating a motion of a plunger that exerts the transdermally injected material.

What is claimed:

1. A transdermal injection component, comprising:
an injection volume configured to contain an injectable treatment therein,
a plunger,
the injection volume being in fluid communication with at least one orifice such that the plunger is configured to exert the injectable treatment from the injection volume through the at least one orifice;
the plunger sealably engaged with the injection volume,
the plunger configured to exert the injectable treatment from the injection volume through the at least one orifice so as to effect transdermal injection of the injectable treatment to a patient;
the transdermal injection component being configured to engage with an actuator device configured to effect movement of the plunger to exert the injectable treatment through the at least one orifice, and any one or more of:
(a) a one-way valve in fluid communication with the sealable injection volume, the one-way valve being configured to place the injection volume into fluid communication with a source of the injectable treatment when the component is engaged with a source of the injectable treatment such that fluid movement is permitted only from the source of the injectable treatment to the injection volume;
(b) a sealer disposed so as to seal the sealable injection volume against an environment exterior to the transdermal injection component when (1) the plunger exerts a negative pressure on the injection volume, (2) the component is engaged with a source of the injectable treatment, or both (1) and (2);
(c) the component defining an opening disposed proximate to the at least one orifice, the opening being dimensioned such that application of a sufficient negative pressure from the opening encourages patient skin toward the opening; and
(d) at least one optionally adjustable element configured to stop a motion of the plunger or a motion of an element engaged with the plunger so as to limit the volume of fluid exerted from the injection volume with motion of the plunger.

2. The transdermal injection component of claim 1, wherein the component comprises a one-way valve in fluid communication with the sealable injection volume, the one-way valve being configured to place the injection volume into fluid communication with a source of the injectable treatment when the component is engaged with the source of the injectable treatment such that fluid movement is permitted only from the source of the injectable treatment to the injection volume.

3. The transdermal injection component of claim 1, wherein the component comprises a sealer disposed so as to seal the sealable injection volume against an environment exterior to the transdermal injection component when (1) the plunger exerts a negative pressure on the injection volume, (2) the component is engaged with a source of the injectable treatment, or both (1) and (2).

4. The transdermal injection component of claim 1, wherein the component comprises an opening disposed proximate to the at least one orifice, the opening being dimensioned such that application of a sufficient negative pressure from the opening encourages patient skin toward the opening.

5. The transdermal injection component of claim 4, wherein the opening extends at least partially circumferentially around the at least one orifice.

6. The transdermal injection component of claim 1, wherein the component defines a plurality of openings disposed proximate to at least one of the at least one orifice, the opening being a first opening of the plurality of openings, the plurality of openings being dimensioned such that application of a sufficient negative pressure from the plurality of openings encourages patient skin toward each of the plurality of openings.

7. The transdermal injection component of claim 1, wherein the transdermal injection component comprises a surface configured to contact a subject, and wherein (a) the at least one orifice stands proud relative to the surface, (b) wherein the at least one orifice stands flush relative to the surface, or (c) wherein the at least one orifice stands recessed relative to the surface.

8. The transdermal injection component of claim 1, wherein the component comprises at least one element configured to stop a motion of the plunger so as to limit the volume of fluid exerted from the injection volume with motion of the plunger.

9. The transdermal injection component of claim 1, wherein the component comprises a plurality of orifices in fluid communication with the sealable injection volume such that the plunger is configured to exert the injectable treatment from the injection volume through the plurality of orifices.

10. The transdermal injection component of claim 1, wherein the injection volume configured to contain an injectable treatment therein is comprised in an ampoule installed in the transdermal injection component.

11. A transdermal injection system, comprising:
a transdermal injection component according to claim 1;
an actuator device configured to engage with the transdermal injection component,
the actuator device comprising a reversibly moveable element configured to effect encouragement of the plunger of the transdermal injection component such that the plunger exerts the injectable treatment through the at least one orifice,
the reversibly moveable element being actuated by operation of an electromagnetic field.

12. The transdermal injection system of claim 11, further comprising a resilient member configured to exert a force on at least one of the reversibly moveable element and the plunger.

13. The transdermal injection system of claim 11, further comprising a source of negative pressure, the source of negative pressure being configured to exert a negative pressure through an opening of the transdermal injection component such that patient skin is exerted generally toward the at least one orifice of the transdermal injection component, optionally such that the patient skin contacts the transdermal injection component.

14. The transdermal injection system of claim 10, further comprising an imaging train, the imaging train being configured to determine one or more features of a patient, a location of the injectable treatment, or both.

15. A method, comprising operating the transdermal injection system of claim 11 so as to effect transdermal injection of the injectable treatment to a patient.

16. A method, comprising:
effecting electromagnetically driven motion of a reversibly moveable element so as to drive a plunger engaged with an injection volume that has an amount of an injectable treatment disposed therein,
the motion effecting exertion of the injectable treatment through one or more orifices in fluid communication with the injection volume so as to effect transdermal injection of the injectable treatment from the one or more orifices to a patient; and
optionally applying a negative pressure to a skin of the patient so as to encourage the skin toward the one or more orifices.

17. The method of claim 16, further comprising effecting a series of transdermal injections, the volume of each of the series of transdermal injections optionally being determined by a movable element disposed within the injection volume.

18. The method of claim 16, further comprising modulating a motion of the plunger in response to a signal collected by an imaging train.

19. A method, comprising:
modulating a flow of a transdermally injected material to a patient in response to a signal collected from an imaging train,
the signal being indicative of a location a blood vessel of the patient, the patient’s epidermis, the patient’s dermis, the patient’s fat, the patient’s muscle, the patient’s bone, or any combination thereof.

20. The method of claim 19, the modulating further being in response to any one or more of a viscosity of the transdermally injected material, a characteristic of the patient’s epidermis, a characteristic of the patient’s dermis, a characteristic of the patient’s fat, a characteristic of the patient’s muscle, and a characteristic of the patient’s bone.
